(19) United States
(12) Patent Application Publication
(10) Pub. No.: US 2025/0392874 A1
(43) Pub. Date: Dec. 25, 2025

(54) METHOD FOR GENERATING TEST SIGNALS TO DETERMINE A HEARING IMPAIRMENT OF A PERSON

(71) Applicant: Baumann UX GmbH, Ingolstadt (DE)

(72) Inventor: Fabian BAUMANN, Ingolstadt (DE)

(73) Assignee: Baumann UX GmbH, Ingolstadt (DE)

(21) Appl. No.: 19/244,360

(22) Filed: Jun. 20, 2025

(30) Foreign Application Priority Data
Jun. 21, 2024 (DE) 10 2024 117 610.5

(51) Int. Cl. H04R 25/00; A61B 5/12

(52) U.S. Cl. CPC H04R 25/505; A61B 5/121; H04R 25/30

(57) ABSTRACT

A method for generating test signals for testing hearing impairment of a person, the method including providing at least one first acoustic signal representing at least one noise; providing at least a second acoustic signal representing speech; for each of the at least one first signal, decomposing the corresponding signal into at least one first frequency band and generating test signals for determining hearing impairment of a person by, for each of the at least one frequency band, respectively superimposing at least a part of the at least one second acoustic signal with the corresponding frequency band, wherein the test signals are generated in such a way, that they respectively comprise only separate components of the at least one noise; and outputting the generated test signals.

### BACKGROUND

### 1. Field of the Disclosure

The disclosure relates to a method for generating test signals for determining a hearing impairment of a person, based on which a hearing solution can be optimally adjusted to the needs of the respective person or the respective patient.

### 2. Background Information

A hearing aid is generally used to output audio signals to a hearing-impaired patient. For this purpose, the hearing aid usually has at least one receiver that converts an audio signal into a sound signal. The hearing aid is regularly worn by the user in or on the ear. Conventional hearing aids change physical, acoustic signal variables in such a way, that a patient fitted with the hearing aid hears better. The hearing aid is adjusted by setting the corresponding parameters or physical transmission variables, such as frequency-dependent amplification, level limitation, etc., until the patient is satisfied within the scope of the possibilities offered.

These parameters are usually set based on the results of a hearing test. It is common practice to first measure the hearing threshold in complete silence, i.e. the volume required per frequency so that the patient can just barely perceive a single tone. In addition, a group of words is played to the patient at normal conversation volume, i.e. 65 dB. The group of words can also be played together with or superimposed on a 60 dB background noise or a disturbance variable, i.e. audiotape noise. However, the disadvantage of this type of method is that it does not provide any information about which noise in particular affects the individual patient and/or when, i.e. in which situations in particular, the individual patient's hearing is impaired during their everyday life.

A hearing aid is known from the document EP 0 674 464 A1, wherein a controller is assigned to an amplifier and transmission part for automatic switching, which, depending on the input variables characterizing the respective environmental situation, makes a selection of the parameter sets stored in the data carrier or of parameters for changing the transmission characteristics of the hearing aid.

A method for generating test signals for testing a person's hearing is known from document DE 10 2007 054 152 A1.

A method for testing the hearing of a small child is known from document US 2020/00000380 A1, wherein an acoustic signal is broken down into individual frequency bands, the individual frequency bands are amplified and the amplified frequency bands are then reassembled so that several frequency ranges can be tested simultaneously.

### SUMMARY

It is therefore an feature of the disclosure to provide a method for generating test signals for determining a person's hearing impairment, based on which a hearing solution can be optimally adjusted to the needs of the respective person or the respective patient.

Advantageous further developments are subject of the dependent claims.

According to one feature of the disclosure, a method is provided for generating test signals for determining a hearing impairment of a person, wherein the method comprises providing at least one first acoustic signal representing at least one noise, providing at least one second acoustic signal representing speech or spoken language, for each of the at least one first signal, decomposing the corresponding signal into at least one first frequency band and generating test signals for determining a hearing impairment of a person by, for each of the at least one first frequency band, respectively superimposing at least a part of the at least one second acoustic signal with the corresponding frequency band, and outputting the generated test signals.

A test signal is a signal that is outputted in order to test a person's or patient's hearing impairment. The test signal can, for example, be composed of natural or spoken speech, such as individual letters or a sequence of words, and a background noise.

An acoustic signal is also understood to be a signal that is reproduced via a sound or noise, such as speech or a disturbing noise.

The fact that the signals are broken down into frequency bands also means that they are broken down or split into individual components, i.e. frequency components or frequency ranges. A frequency range is understood to be a limited, contiguous section or range of these frequency components.

The fact that the signals are then superimposed also means that they are added up or superimposed.

The method thus generates test signals that only contain individual parts or components of a noise, whereby the test signals can then be used in particular to find out which of these components actually impair the hearing of the respective person. Based on the generated test signals, realistic hearing situations with a variable degree of difficulty, i.e. hearing situations that can be controlled or broken down into their individual components, can be simulated, making it easier to simulate everyday impairments.

Overall, a method for generating test signals to determine a person's hearing impairment is thus provided, based on which a hearing solution can be optimally adjusted to the needs of the respective person or patient.

In one feature, the method further comprises a step of, for each of the at least one second signal, respectively decomposing the corresponding signal into at least one second frequency band, wherein the test signals for determining a hearing impairment of a person are generated by respectively superimposing each of the at least one first frequency band with one or more of the at least one second frequency band. In this way, the speech signal or the spoken language can also be broken down into individual components, for example individual letters, so that the hearing ability of the person in question can be determined even more precisely.

The at least one first frequency band and/or the at least one second frequency band can comprise frequency bands with different widths.

The width of a frequency corresponds to the frequency range covered by it.

Based on frequency bands with different widths, a corresponding hearing test can be further refined and/or made progressively more difficult, whereby the hearing ability or hearing impairment of the respective person can be determined even more precisely, in particular which noises or sounds actually affect the hearing ability of the respective person.

According to a further feature of the disclosure, also a method for determining a hearing impairment of a person is provided, the method comprising generating test signals for determining a hearing impairment of the person by a method for generating test signals for determining a hearing impairment of a person as described above and determining a hearing impairment of the person based on the generated test signals.

Thus, a method for determining a person's hearing impairment is provided, which is based on test signals, based on which a hearing solution can be optimally adjusted to the needs of the respective person or the respective patient. In particular, the method is based on test signals which only contain individual components of a noise, whereby the test signals can be used in particular to find out which of these components actually impairs the hearing of the respective person. Based on the generated test signals, realistic hearing situations with a variable degree of difficulty, i.e. hearing situations that can be controlled or broken down into their individual components, can be simulated, which means that everyday impairments can be better simulated.

The step of determining a hearing impairment of the person based on the generated test signals may comprise a simulation of different compensation types for hearing loss and a determination of the hearing impairment for at least two of the different compensation types.

Compensation for hearing loss refers to the way in which background noise can be processed, for example with the support of a hearing aid, or the way in which it can be responded to them. For example, background noise can be completely filtered out or sounds that are detected by the processing in the hearing aid can be changed as little as possible.

In addition to determining the optimal hearing ability, this also allows to determine optimal compensation methods for the respective patient, which enables an improved pre-selection of hearing solutions or hearing aids suitable for the respective patient.

According to a further feature of the disclosure, also a method for adjusting parameters of a hearing aid of a person is provided, the method comprising determining a hearing impairment of the person by a method for determining a hearing impairment of a person as described above and adjusting parameters of the hearing aid based on the hearing impairment of the person.

The parameters of a hearing aid are understood to be the transmission and/or amplification functions or sizes of the hearing aid.

Thus, a method for adjusting a hearing aid is provided, which is based on test signals, based on which a hearing solution can be optimally adjusted to the needs of the respective person or the respective patient. In particular, the method is based on test signals which only contain individual components of a noise, whereby the test signals can be used in particular to find out which of these components actually impair the hearing of the respective person. Based on the generated test signals, realistic hearing situations with a variable degree of difficulty, i.e. hearing situations that can be controlled or broken down into their individual components, can be simulated, which means that everyday impairments can be better simulated.

According to a further feature of the disclosure, also a device for generating test signals for determining a hearing impairment of a person is provided, the device comprising a first providing unit adapted to provide at least one first acoustic signal representing at least one noise, a second providing unit adapted to provide at least one second acoustic signal representing speech, and a generation unit, which is configured to, for each of the at least one first signal, split the corresponding signal into at least one first frequency band and to generate test signals for testing the hearing ability of a person by, for each of the at least one first frequency band, superimposing at least a part of the at least one second acoustic signal with the corresponding frequency band, and an output unit, which is designed to output the generated test signals.

Thus, a device for generating test signals for determining a person's hearing impairment is provided, based on which a hearing solution can be optimally adjusted to the needs of the respective person or the respective patient. In particular, the device generates test signals which only contain individual components of a noise, whereby the test signals can then be used in particular to find out which of these components actually impairs the hearing of the respective person. Based on the generated test signals, realistic hearing situations with a variable degree of difficulty, i.e. hearing situations that can be controlled or broken down into their individual components, can be simulated, making it easier to simulate everyday impairments.

In one feature, the generation unit is further configured to, for each of the at least one second signal, decompose the corresponding signal into at least one second frequency band and to generate the test signals for determining a person's hearing impairment by superimposing one or more of the at least one second frequency band on each of the at least one first frequency band. In this way, the speech signal or the spoken language can also be broken down into individual components, for example individual letters, so that the hearing ability of the person in question can be determined even more precisely.

The at least one first frequency band and/or the at least one second frequency band can comprise frequency bands with different widths. Based on frequency bands with different widths, a corresponding hearing test can be further refined, for example made progressively more difficult, whereby the hearing ability of the corresponding person can be determined even more precisely, in particular which noises or sounds actually influence the hearing ability of the corresponding person.

According to a further feature of the disclosure, also a system for detecting a hearing impairment of a person is provided, the system comprising a device for generating test signals for detecting a hearing impairment of a person as described above and a test unit adapted to test the hearing impairment of the person based on test signals generated by the device for generating test signals for detecting the hearing impairment of a person.

Thus, a system for determining a person's hearing impairment is provided, which is based on test signals on the basis of which a hearing solution can be optimally adjusted to the needs of the respective person or the respective patient. In particular, the system is based on test signals which only contain individual components of a noise, whereby the test signals can be used in particular to find out which of these components actually impairs the hearing of the respective person. Based on the generated test signals, realistic hearing situations with a variable degree of difficulty, i.e. hearing situations that can be controlled or broken down into their individual components, can be simulated, making it easier to simulate everyday impairments.

The test unit can be configured to simulate different types of compensation for hearing loss and to determine the hearing impairment for at least two of the different types of compensation. In this way, in addition to determining the optimum hearing ability, optimum compensation methods can also be determined for the corresponding patient, which enables an improved preselection of hearing solutions or hearing aids suitable for the corresponding patient.

According to a further feature of the disclosure, also a system for adjusting parameters of a hearing aid of a person is provided, the system comprising a system for determining a hearing impairment of a person as described above and an adjustment unit adapted to adjust parameters of the hearing aid based on the hearing impairment of the person determined by the system for determining a hearing impairment of a person.

Thus, a system for adjusting a hearing aid is provided, which is based on test signals, based on which a hearing solution can be optimally adjusted to the needs of the respective person or the respective patient. In particular, the system is based on test signals which only contain individual components of a noise, whereby the test signals can be used in particular to find out which of these components actually impair the hearing of the respective person. Based on the generated test signals, realistic hearing situations with a variable degree of difficulty, i.e. hearing situations that can be controlled or broken down into their individual components, can be simulated, making it easier to simulate everyday impairments.

According to a further feature of the disclosure, also a computer program comprising program code to execute a method for generating test signals for determining a hearing impairment of a person as described above when the computer program is executed on a computer is provided.

The computer program has the advantage that it is designed to carry out a method for generating test signals for determining a person's hearing impairment, based on which a hearing solution can be optimally adjusted to the needs of the respective person or patient. In particular, the method generates test signals which only contain individual parts or components of a noise, whereby the test signals can then be used in particular to find out which of these components actually impairs the hearing of the respective person. Based on the generated test signals, realistic hearing situations with a variable degree of difficulty, i.e. hearing situations that can be controlled or broken down into their individual components, can be simulated, making it easier to simulate everyday impairments.

In summary, the present disclosure provides a method for generating test signals for determining a hearing impairment of a person, based on which a hearing solution can be optimally adjusted to the needs of the respective person or the respective patient.

### BRIEF DESCRIPTION OF THE DRAWINGS

The disclosure will now be explained in more detail with reference to the attached figures.

FIG. 1. illustrates a flowchart of a method for determining a hearing impairment of a person according to features of the disclosure;

FIG. 2. illustrates a schematic block diagram of a system for determining a hearing impairment of a person according to features of the disclosure.

### DETAILED DESCRIPTION

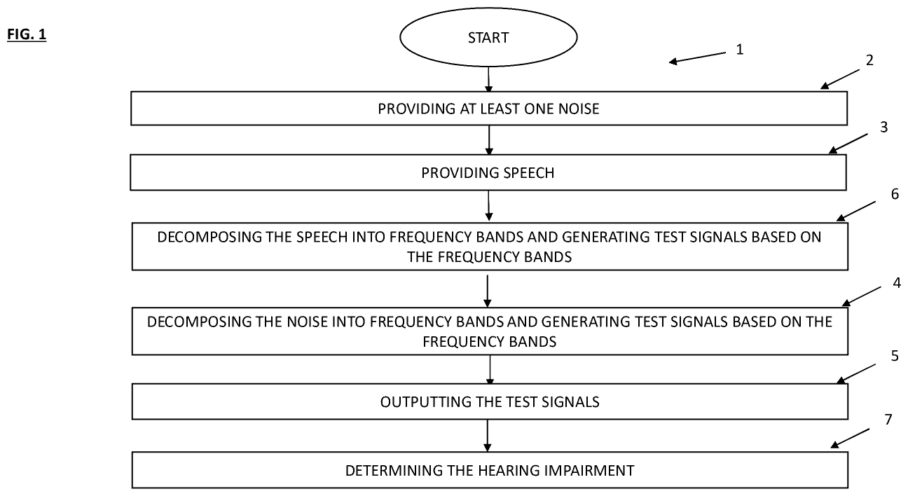

FIG. 1 illustrates a flowchart of a method for determining a hearing impairment of a person 1 according to features of the disclosure.

The first point of contact when fitting a hearing aid is usually the hearing test. The hearing threshold, i.e. the volume required per frequency for a patient to just be able to perceive a single tone, is first measured in complete silence. In addition, a phrase is played to the patient at a normal conversational volume, usually 65 dB, which the patient is then asked to repeat. If the patient is unable to repeat more than 80% of the word group correctly, the volume of the reproduced speech signals is usually increased gradually, broadband, i.e. without frequency adjustment, until the patient understands as well as possible or feels discomfort, which is represented by the discomfort threshold.

However, this measurement procedure does not take everyday situations into account. For example, most hearing problems first occur in noise or when additional background noise occurs, which means that the mental effort required to cope in a social situation in which speech and background noise occur together must be increased.

If the group of words described above is reproduced in 65 dB with an additional 60 dB of background noise, restrictions caused by the background noise, for example brandband noise, can be made clear. Although this measurement already comes close to the perceived hearing problem in noise, it is not possible to simulate a direct improvement. This measurement, with and without background noise, is usually only used to conclusively prove the resulting improvement of a hearing solution for the health insurance subsidy.

Regardless of the used test signal or the corresponding objective, the hearing aid itself is also fitted without the presence of background noise, as this is the only way to record the gain curve of the hearing aid without superimposition. However, an additional signal that represents the background noise of a real situation is not currently used to fine-tune the hearing system.

Each hearing aid also has a dynamic transmission characteristic developed by the manufacturer, which is mapped in individual features of the system but cannot be seen from the outside. Hearing systems are no longer pure amplifiers, but are based on an internal algorithm that changes the transmission depending on the detected situation. This behavior can only be changed or compared to a limited extent by the hearing care professional fitting. Fitting procedures are static and restore the patient's hearing ability in quiet and not the maximum possible benefit and comfort with active features in noise.

As FIG. 1 illustrates, the method 1 comprises a step 2 of providing at least one first acoustic signal representing at least one noise, a step 3 of providing at least one second acoustic signal representing a speech, for each of the at least one first signal, a step 4 of decomposing the corresponding signal into at least one first frequency band and generating test signals for determining a hearing impairment of a person by, for each of the at least one first frequency band, respectively superimposing at least a part of the at least one second acoustic signal with the corresponding frequency band, and a step 5 of outputting the generated test signals.

Test signals are generated by the method 1, each of which has only individual constituents or components of a noise, whereby the test signals can then be used in particular to find out which of these components actually impairs the hearing ability of the respective person. Based on the generated test signals, realistic hearing situations with a variable degree of difficulty, i.e. hearing situations that can be controlled or broken down into their individual components, can be simulated, which means that everyday impairments can be better simulated.

Overall, a method for generating test signals for determining a hearing impairment of a person 1 is thus provided, based on which a hearing solution can be optimally adjusted to the needs of the respective person or the respective patient.

In particular, test signals can be generated based on which known situations, such as a visit to a restaurant, can be presented or reproduced. Based on these test signals, hearing impairments that actually occur in the patient's everyday life can be recorded or it can be determined when losses of clarity actually occur.

According to the features of FIG. 1, the method 1 further comprises a step 6 of, for each of the at least one second signal, decomposing the at least one second signal into at least one second frequency band, wherein the test signals for testing the hearing ability of a person are generated in step 4 by respectively superimposing one or more of the at least one second frequency band on each of the at least one first frequency band.

A spectral analysis of the speech signal or the spoken signal is therefore also carried out.

For example, each of the first frequency bands can be successively overlaid with each of the second frequency bands.

Based on corresponding test signals, it is then possible to check, for example, how low background noise should be reduced in different bands for an individual patient and how high speech should be raised in individual bands accordingly.

According to the features of FIG. 1, the at least one first frequency band and the at least one second frequency band each have at least two frequency bands with different widths.

The width can vary, for example, between frequency bands comprising complete letter sequences, frequency bands comprising 1 kHz or one log atom and frequency bands comprising just one letter.

Based on the different widths, changes in the individual frequency bands can be reproduced in several steps and widths, which can for example be used to simulate gradual equalization when setting parameters of a hearing aid. For example, a letter-to-noise ratio can be adjusted during a hearing test until the corresponding letter is understood.

As FIG. 1 shows, the method 1 also comprises a step 7 of determining a hearing impairment of a person or patient based on the generated test signals.

In particular, the signal-to-noise ratio required in corresponding situations, for example the signal-to-noise ratio per letter, can be determined based on the individual test signals.

Compensation and potential improvement of frequency-dependent hearing can be accurately simulated in noisy situations, especially in everyday situations.

The method 1 can also be used to simulate masking effects and gradual compensation of hearing loss.

The fact that hearing impairments and not physical hearing loss are determined also has the advantage that age and the age-dependent ability to understand or reproduce sounds in the brain are taken into account. For example, a 30-year-old usually has fewer limitations with the same hearing loss than a 70-year-old with limited brain activity.

The results of the hearing test can for example be recorded in the form of a matrix.

According to the features of FIG. 1, the step 7 of testing the person's hearing based on the generated test signals further comprises simulating different compensation types for hearing loss and determining the hearing impairment for at least two of the different compensation types.

In particular, it simulates what the hearing aid can theoretically do.

For example, wind noise suppression and/or varying degrees of noise suppression can be simulated.

Based on the test results, hearing aid parameters can then for example be adjusted or approximated.

In particular, the more precise results obtained support the decision for a hearing aid and also the selection of optimal compensation strategies.

For example, the control depth can be individually adjusted, i.e. how strongly the device should regulate or the volume should be adjusted accordingly.

The method 1 thus enables the appropriate combination of processing strategy, amplifier settings and technical equipment to be found and implemented for the individual patient.

In particular, the process steps can be carried out before a hearing aid is purchased. In addition, the procedure can also be carried out dynamically, i.e. while wearing a hearing aid, whereby the parameters of the hearing aid can be readjusted or optimized based on the corresponding test results.

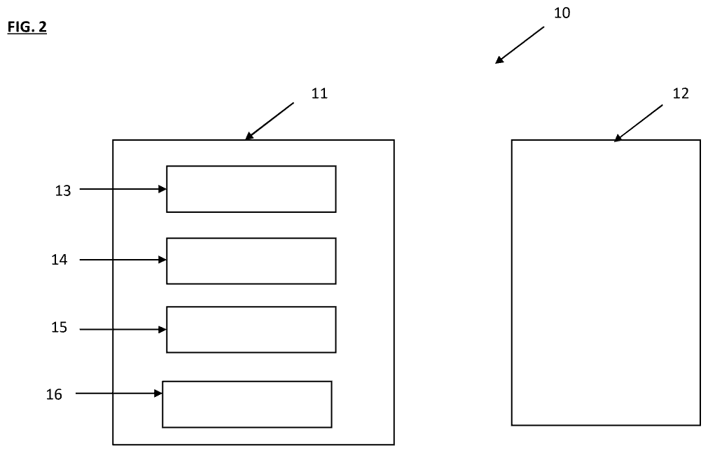

FIG. 2 illustrates a schematic block diagram of a system for determining a hearing impairment of a person 10 according to features of the disclosure.

As FIG. 2 shows, the illustrated system 10 comprises a device for generating test signals for determining a hearing impairment of a person 11 and a test unit 12 which is configured to test a hearing impairment of the person based on test signals generated by the device for generating test signals for determining a hearing impairment of a person.

The test unit can be based on audio output units and corresponding computing units and can be configured to test the person's hearing impairment in corresponding frequency ranges by means of sound and speech audiometry.

The illustrated device 11 further comprises a first providing unit 12, which is configured to provide at least one first acoustic signal representing at least one noise, a second providing unit 13, which is configured to provide at least one second acoustic signal representing speech, a generation unit 14, which is configured to, for each of the at least one first signal, decompose the corresponding signal into at least one first frequency band and to generate test signals for determining a hearing impairment of a person by, for each of the at least one first frequency band, respectively superimposing at least a part of the at least one second acoustic signal with the corresponding frequency band, and an output unit 15 which is configured to output the generated test signals.

In particular, the first and second providing units may be receivers that are configured to receive corresponding audio signals, whereby the first and second providing units may also each comprise a microphone to record acoustic signals.

The generation unit can also be realized, for example, based on a code that is stored in a memory and executable by a processor.

The output unit can also be an audio output unit such as a loudspeaker.

According to the features of FIG. 2, the generating unit 14 is further configured to, for each of the at least one second signal, decompose the corresponding signal to into at least one second frequency band and to generate the test signals for testing the hearing ability of a person by successively superimposing one or more of the at least one second frequency band on each of the at least one first frequency band.

In addition, the at least one first frequency band and the at least one second frequency band again respectively comprise at least two frequency bands with different widths.

According to the features of FIG. 2, the test unit 12 is also configured to simulate different types of compensation for hearing loss and to determine the hearing impairment for at least two of the different types of compensation.

The shown system is also configured to execute a method for determining hearing impairment of a person as described.

1. A method for generating test signals for testing hearing impairment of a person, the method comprising:
providing at least one first acoustic signal representing at least one noise;
providing at least a second acoustic signal representing speech;
for each of the at least one first signal, decomposing a respective said acoustic signal into at least one first frequency band and generating test signals for determining hearing impairment of a person by, for each of the at least one frequency band, respectively superimposing at least a part of the at least one second acoustic signal with a corresponding said frequency band, wherein the test signals are generated in such a way, that they respectively comprise only separate components of the at least one noise; and
outputting the generated test signals.

2. The method according to claim 1, further comprising, for each of the at least one second acoustic signal, decomposing the at least one second acoustic signal into at least one second frequency band, wherein the test signals for determining hearing impairment of a person are generated by respectively superimposing each of the at least one first frequency band with one or more of the at least one second frequency band.

3. The method according to claim 1, wherein the at least one first frequency band or the at least one second frequency band comprises frequency bands of different widths.

4. A method for determining hearing impairment of a person, the method comprising:
generating test signals for testing hearing impairment of the person by a method for generating test signals for testing hearing impairment of a person according to claim 1; and
determining hearing impairment of the person based on the generated test signals.

5. The method according to claim 4, wherein the determining of hearing impairment of the person based on the generated test signals comprises simulating different compensation types for hearing loss and determining hearing impairment for at least two of the different compensation types.

6. A method of adjusting parameters of hearing aid of a person, the method comprising:
determining hearing impairment of the person by the method for determining hearing impairment of a person according to claim 4; and
setting the parameters of the hearing aid based on the hearing impairment of the person.

7. A device for generating test signals for determining hearing impairment of a person, the device comprising:
a first receiver configured to provide at least one first acoustic signal representing at least one noise;
a second receiver configured to provide at least one second acoustic signal representing speech;
a generator configured to, for each of the at least one first signal, to decompose the corresponding signal into at least one first frequency band and to generate test signals for determining hearing impairment of a person by, for each of the at least one first frequency band, respectively superimposing at least a part of the at least one second acoustic signal with the corresponding frequency band, wherein the test signals are generated in such a way, that they respectively comprise only separate components of the at least one interfering noise; and
an output configured to output the generated test signals.

8. The device according to claim 7, wherein the generator is further configured to, for each of the at least one second acoustic signal, decompose the corresponding signal into at least one second frequency band and to generate the test signals for determining hearing impairment of a person by respectively superimposing each of the at least one first frequency band with one or more of the at least one second frequency band.

9. The device according to claim 7, wherein the at least one first frequency band and the at least one second frequency band comprises frequency bands with different widths.

10. A system for determining hearing impairment of a person, the system comprising:
a test signal generator for the device for generating test signals for determining hearing impairment of a person according to claim 7; and
a test processor configured to determine the hearing impairment of the person based on test signals generated by the device for generating test signals for determining hearing impairment of a person.

11. The system according to claim 10, wherein the test processor is configured to simulate different types of compensation for hearing loss and to determine the hearing impairment for at least two of the different types of compensation.

12. A system for adjusting parameters of hearing aid of a person, the system comprising:
a system for determining hearing impairment of a person according to claim 10; and
an adjuster configured to adjust parameters of the hearing aid based on the hearing impairment of the person determined by the system a system for determining hearing impairment of a person.

13. A non-transitory computer-readable medium storing at least one computer program for causing a computer to execute processing comprising the method of generating test signals for testing hearing impairment of a person according to claim 1.
